(19) United States
(12) Patent Application Publication
(10) Pub. No.: US 2024/0399656 A1
(43) Pub. Date: Dec. 5, 2024

(54) METHOD AND DEVICE FOR PRODUCING A COATED OBJECT BY MEANS OF ADDITIVE MANUFACTURING IN A VACUUM

(71) Applicant: Universität der Bundeswehr München, Neubiberg (DE)

(72) Inventors: Kilian MÜLLER, Munich (DE); Petra MELA, Garching bei München (DE); Markus EBLENKAMP, Höhenkirchen-Siegertsbrunn (DE); Nicolas NIESSEN, München (DE); Marina KÜHN-KAUFFELDT, München (DE); Marvin KÜHN, München (DE)

(21) Appl. No.: 18/292,707

(22) PCT Filed: Jul. 29, 2022
(86) PCT No.: PCT/EP2022/071430

§ 371 Date: Jan. 26, 2024

(30) Foreign Application Priority Data
Jul. 30, 2021 (DE) 102021119926.3

(51) Int. Cl. B29C 64/159; B29C 64/118; B29C 64/227; B29C 64/364; B29K 71/00; B29L 31/00; B33Y 10/00; B33Y 30/00; C23C 14/04

(52) U.S. Cl. CPC B29C 64/159; B29C 64/118; B29C 64/227; B29C 64/364; B33Y 10/00; B33Y 30/00; C23C 14/04; B29K 2071/00; B29L 2031/7532

(57) ABSTRACT

The present invention relates to a method for producing a coated object by additive manufacturing in vacuum and to a device for producing a coated object. The method comprises forming a first polymer layer on a workpiece and/or on a printing bed. A first coating layer is formed on at least a part of the first polymer layer by vacuum-based vapor deposition. A second polymer layer is formed on at least a part of the first polymer layer and/or of the first coating layer, wherein the second polymer layer is formed before, during or after forming the first coating layer. The first polymer layer, the first coating layer and the second polymer layer are formed in vacuum at a gas pressure of less than 10⁻² mbar.

### FIELD OF THE INVENTION

The present invention is in the field of additive manufacturing and relates to a method and a device for producing a coated object by additive manufacturing in vacuum.

### BACKGROUND

Polymer materials, in particular high-performance thermoplastics, have a variety of advantageous properties for the production of objects with complex shapes such as medical implants, see e.g. R. F. M. R. Kersten et al., Spine J. 15 (2015) 1446-1460 and S. M. Kurtz, PEEK Biomaterials Handbook, 2nd edition, William Andrew, Oxford, 2019. Many thermoplastic materials such as polymers of the polyaryletherketone group (PAEK group) are biocompatible and have mechanical characteristics similar to those of human bone. High-performance plastics are also of interest for other applications, for example in aerospace, due to their thermal and chemical durability as well as their high mechanical stability and their low weight. In addition, thermoplastic materials are generally easy to process and thus enable the production of individually adapted objects such as bone replacement implants. In particular, they are suitable for additive manufacturing (3D printing), in which items are produced by iteratively forming a plurality of layers, whereby even complex structures can be produced quickly and cost-efficiently.

For some applications, however, polymer materials can be unsuitable in particular as surface material and require additional functionalization. Polymer materials are generally bioinert, for example, which makes, e.g., the growing of bone structures on such implants considerably more difficult. In order to nonetheless enable the growing of bone structures, the surface of the finished implant can be modified, for example by applying an osseointegrative coating or by microstructuring the surface to increase its roughness, see e.g. J. Knaus et al., Macromol. Biosci. 20 (2020) 1900239. Moreover, most polymer materials are electrical insulators, so that, for example, a metallic coating has to be applied in order to obtain an electrically conductive surface.

Such a surface modification usually takes place in an additional processing step downstream of the production of the polymeric base body. In addition to the higher complexity and costs of production associated therewith, this also entails the risk of contamination, in particular in the case of medical implants, and therefore requires special measures in order to ensure the sterility of the implant. Moreover, objects with complex geometries such as undercuts or porous structures cannot be coated with conventional coating methods. As a result, the freedom in terms of design made possible by the additive manufacturing in the first place is at least partially restricted again.

### SUMMARY

It is therefore an object of the invention to enable the production of objects with complex structures and functionalized surfaces in order to facilitate, e.g., the growing of bone structures on medical implants.

This object is met according to the invention by a method for producing a coated object according to claim 1 and a device according to claim 11. Examples of the invention are defined in the dependent claims.

The method according to the invention relates to the production of a coated object by additive manufacturing in vacuum. The method comprises forming a first polymer layer on a workpiece and/or on a printing bed. A first coating layer is formed on at least a part of the first polymer layer by vacuum-based vapor deposition. In addition, a second polymer layer is formed on at least a part of the first polymer layer and/or of the first coating layer, wherein the second polymer layer is formed before, during or after forming the first coating layer. The first polymer layer, the first coating layer and the second polymer layer are formed in vacuum at a gas pressure of less than 1 mbar.

The coated object can be, e.g., a medical implant, in particular a bone replacement implant, i.e. an implant which serves to completely or partially replace, repair or enlarge a bone in the human or animal body, e.g. after tumor resection or removal of the original bone due to debris fracture. The implant can be, e.g., an endoprosthesis or a part of an endoprosthesis which serves to replace a joint completely or partially. In other embodiments, the implant can be, e.g., a dental implant. The method according to the invention can be used, e.g., to provide the medical implant with an osseointegrative and/or antibacterial coating. In some embodiments, the coated object can also be intended for use in aerospace. The object can be, e.g., a component of an aircraft, e.g. a motor component, and can be provided with a functionalized surface, e.g. an electrically and/or thermally conductive surface, using the method according to the invention. In another example, the object can be a component of a satellite or a space probe, e.g. a component for a solar panel or an antenna, and, using the method according to the invention, can be provided with a functionalized surface, e.g. with a radiation-resistant surface, e.g. made of aluminum, for protection against ultraviolet or cosmic radiation, a surface for protection against atomic oxygen or a surface with improved thermal conductivity. The method according to the invention can in particular also be used for producing replacement parts in orbit.

The coated object is produced at least partially using an additive manufacturing method, also referred to as 3D printing, e.g. with the device according to the invention. The coated object can be produced, e.g., from a plurality of layers which are successively formed one above the other. The expression “formed one above the other” is also intended to include those embodiments in which a first layer and the layer following the first layer are not in contact with one another over their entire areas and/or a first layer and the layer following the first layer—as viewed in the build-up direction—are not entirely congruent, in particular do not have an identical outer contour. “Formed one above the other” rather indicates that, according to the method of production, a build-up direction is provided along which the coated object or workpiece is successively built from a plurality of layers and which extends substantially along the normal vector of the layers, preferably along the vertical direction. The layers can be formed, e.g., by depositing a liquid or liquefied material such as a molten thermoplastic material, which subsequently solidifies or hardens. Preferably, the coated object is produced by fused filament fabrication (fused deposition modeling).

The workpiece can be provided at the beginning of the method according to the invention or can be formed as part of the method according to the invention. A workpiece, as used herein, refers to the object to be produced in an intermediate stage at the beginning or during the production of the coated object, e.g., at the beginning of or during the method according to the invention. In particular, the workpiece can be produced entirely or partially using an additive manufacturing method, e.g., from plastic, ceramic and/or metal, e.g., by forming a plurality of polymer and/or coating layers before forming the first polymer layer. Alternatively or additionally, the workpiece can also be produced, e.g., by using a milling and/or injection molding method.

First, the first polymer layer is formed on the workpiece, e.g., by depositing a polymer material in liquid form and subsequent solidification or hardening. The first polymer layer can be formed on the entire workpiece or preferably selectively on one or more regions of the workpiece, e.g., according to a predetermined shape of the coated object, e.g., based on a construction model of the coated object such as a CAD model.

Subsequently, a first coating layer is formed on at least a part of the first polymer layer, e.g., on at least a part of a top surface of the first polymer layer that is substantially perpendicular to the build-up direction and/or on at least a part of a side surface of the first polymer layer that is substantially parallel to the build-up direction. The first coating layer can comprise, e.g., an inorganic, thermally conductive and/or electrically conductive material, in particular a metal, or can consist of such a material. The first coating layer can comprise, e.g., titanium, aluminum, copper, silicon, carbon, nitrogen, oxygen and/or fluorine. A thermally conductive material can be, e.g., a material having a thermal conductivity of more than 10 W/(m·K), preferably having a thermal conductivity of more than 50 W/(m·K), in one example having a thermal conductivity of more than 100 W/(m·K). An electrically conductive material can be, e.g., a material having an electrical conductivity of more than 10² (Ω·m)⁻¹, preferably having an electrical conductivity of more than 10⁴ (Ω·m)⁻¹, in one example having an electrical conductivity of more than 10⁶ (Ω·m)⁻¹.

In some embodiments, the coated object is a medical implant, e.g. a bone replacement implant, and the first coating layer comprises or consists of an osseointegrative and/or antibacterial material. An osseointegrative material is a material on which bone tissue can grow. The osseointegrative material can be configured, e.g., such that osteoblasts adhere to its surface. The osseointegrative material can comprise an osseointegrative metal, preferably titanium, and can be, e.g., pure titanium, titanium oxide and/or a titanium alloy. An antibacterial material is a material which inhibits or completely prevents the adhesion, growth and/or reproduction of bacteria and/or kills bacteria in a targeted manner, e.g. a material toxic to bacteria. The antibacterial material can comprise an antibacterial metal, preferably copper, silver and/or aluminum, and can be, e.g., pure copper, copper oxide, a copper alloy, pure silver, silver oxide and/or a silver alloy. Alternatively or additionally, the antibacterial material can also comprise titanium, in particular titanium dioxide (TiO₂). Titanium dioxide can have an antibacterial effect, e.g., by irradiation with light (photocatalysis), in particular UV light.

The first coating layer is formed by a vacuum-based vapor deposition method in which the coating material or a precursor material is brought into the vapor phase in a vacuum environment and deposited on the workpiece and/or on the first polymer layer. The first coating layer can be formed, e.g., by chemical vapor deposition or preferably by physical vapor deposition. The coating material can be vaporized, e.g., thermally or by a laser beam and subsequently condense on the workpiece and the first polymer layer, respectively. In a preferred embodiment, the first coating layer is formed by cathodic arc deposition (Arc-PVD). For this, a gas discharge can, e.g., be induced by applying a voltage to a target made of an electrically conductive coating material, and coating material from the target can be brought into the vapor phase by the resulting plasma.

Furthermore, a second polymer layer is formed on the workpiece, wherein the second polymer layer can be formed on at least a part of the first polymer layer and/or on at least a part of the first coating layer. The second polymer layer can be formed after or during forming the first polymer layer and before, during or after forming the first coating layer. The first coating layer can be formed, in terms of time, between the first and second polymer layers, e.g., after completing the formation of the first polymer layer and before beginning the formation of the second polymer layer. In other examples, the first coating layer can be formed after forming the first and second polymer layers, e.g., as the last layer of the additive manufacturing process. In some embodiments, the first polymer layer, the first coating layer and/or the second polymer layer can be formed simultaneously at least in part. For example, the second polymer layer can already be formed in a first region of the workpiece, while the first coating layer is still being formed in a second region of the workpiece. In other embodiments, the second polymer layer can only be formed when the formation of the first coating layer has already been completed.

The first coating layer as well as the first polymer layer and the second polymer layer are formed in vacuum. The workpiece can be located, for example, in a vacuum chamber during the method. The gas pressure during the formation of the layers is less than 1 mbar (medium/fine vacuum). In some examples, the gas pressure is less than 10⁻¹ mbar, preferably less than 10⁻² mbar. In some embodiments, the layers are formed in high vacuum (≤10⁻³ mbar), in one example at a gas pressure of less than 10⁻⁴ mbar. In some embodiments, the production of the workpiece and/or the further production of the coated object also takes place in vacuum, for example by forming a plurality of polymer and/or coating layers in vacuum at the corresponding gas pressure.

The first coating layer can be formed on the entire first polymer layer and/or on the entire workpiece, e.g., as a homogeneous layer. In other embodiments, the first coating layer can be formed only on a part of the first polymer layer, e.g., only on regions of the first polymer layer which form an exposed surface of the coated object after completion of the production of the coated object. This can be advantageous, e.g., for the mechanical stability of the coated object, but at the same time enable functionalization of the surface, e.g., in order to enable the growing of bone structures on a bone replacement implant. The exposed surface can be exposed to the outside, i.e., be in contact with an environment of the coated object. Alternatively or additionally, the exposed surface can also be exposed to the inside, i.e., for example be a surface bordering a cavity within the coated object. In other embodiments, the first coating layer can be completely or partially enclosed by polymer layers after completion of the production of the coated object, e.g., in order to provide the first polymer layer in the interior of the object with a functionalized coating, for example with an electrically conductive structure.

In some embodiments, forming the first coating layer can comprise adding a process gas, e.g., to form a reactive coating. The process gas can be added before, during or after the vapor deposition, e.g., to introduce a further species, in particular a reactive species, into the first coating layer or to form a further layer, in particular a reactive layer, under or on the first coating layer. The process gas can comprise, e.g., nitrogen, oxygen, argon, water vapor, methane and/or carbon dioxide.

The first polymer layer and the second polymer layer can comprise a thermoplastic material or can consist of a thermoplastic material. Preferably, the first and second polymer layers each comprise or consist of one or more engineering polymers and/or of one or more high-performance polymers. The group of engineering polymers comprises, inter alia, acrylonitrile-butadiene-styrene (ABS), nylon 6, nylon 6-6, polyamide (PA), polybutylene terephthalate (PBT), polycarbonate (PC), polyketone (PK), polyethylene terephthalate (PET), polyimide, polyoxymethylene (POM/acetal), polyphenylene oxide (PPO) and polymethyl methacrylates (PMMA). The group of high-performance polymers comprises, inter alia, polyphenylene sulfide (PPS), polysulphone (PSU), polytetrafluoroethylene (PTFE/Teflon) and the group of polyaryletherketones (PAEK) including polyetheretherketone (PEEK), polyetherketoneketone (PEKK) and polyetherketone (PEK). The polymer layers can each comprise, e.g., one or more polyetherketones (PEK), preferably polyaryletherketones (PAEK), in particular polyetheretherketone (PEEK). The first and second polymer layers can consist of the same polymer material or of different polymer materials. In some embodiments, the first and/or second polymer layers can alternatively or additionally also consist of one or more biodegradable polymers or comprise such materials. A biodegradable polymer is a polymer which is biologically degradable and can be completely or partially degraded in the human body, e.g., over a period of several weeks, several months or several years. The group of biodegradable polymers comprises, e.g., polycaprolactone (PCL), polyglycolides (PGA), polylactides (PLA), polyethylene glycol (PEG) and polydioxanone (PDS).

The first polymer layer and the second polymer layer can each be formed by fused filament fabrication (fused deposition modeling). In this case, a filament or strand of a thermoplastic material is completely or partially melted and selectively deposited on the workpiece, e.g., along a predefined path, in order to form a structured polymer layer. The filament can be fed to a movably supported print head, melted therein and deposited on the workpiece through a nozzle. Basic principles of additive manufacturing by fused filament fabrication are described, e.g., in S. Vyavahare et al., Rapid Prototyp. J. 26 (2020), 176-201, B. N. Turner et al., Rapid Prototyp. J. 20 (2014), 192-204 and B. N. Turner/S. A. Gold, Rapid Prototyp. J. 21 (2015), 250-261.

The method according to the invention can further comprise forming further polymer layers and/or further coating layers, e.g., before forming the first polymer layer and/or after forming the second polymer layer. In particular, the method can comprise additively manufacturing the workpiece by iteratively forming a plurality of polymer and/or coating layers and/or finishing the coated object by iteratively forming a plurality of further polymer and/or coating layers on the workpiece. Each of the coating layers can be deposited, by vacuum-based vapor deposition, on at least a part of an already formed polymer layer and before forming a further polymer layer.

In some embodiments, the method comprises iteratively forming a plurality of polymer layers and a plurality of coating layers, wherein the polymer layers and the coating layers are formed alternatingly, e.g., by forming a coating layer between the formation of two successive polymer layers. In other embodiments, a different sequence of layers can be selected, e.g., by forming a respective coating layer after forming a predetermined number of polymer layers, wherein the predetermined number of polymer layers can be between 2 and 10, for example. In another embodiment, the method comprises iteratively forming a plurality of polymer layers, wherein only a single coating layer, namely the first coating layer, is formed, e.g., after forming the last polymer layer of the plurality of polymer layers.

The method according to the invention enables the production of coated objects with complex structures, e.g., of medical implants with a functionalized surface that facilitates the growing of bone structures thereon. By forming the first coating layer together with the first polymer layer and the second polymer layer in a vacuum, e.g., by forming the first coating layer between forming the first and second polymer layers or after forming the first and second polymer layers, the coating of the object can be integrated into the additive manufacturing process. Thereby, a downstream coating process is not necessary, for example, even in the case of implants made of bioinert polymers, whereby, in addition to simplifying the production process, the risk of contamination can also be reduced. In addition, a complete and uniform coating can also be achieved in the case of complex structures, e.g., of cavities, pores or undercuts, or a coating of polymer layers which are located in the interior of the object, i.e., which are completely or partially enclosed by other polymer layers after production. The entire method is carried out in a medium/fine or high vacuum, whereby a contamination-free manufacturing process can be ensured without a clean room or a particle-filtered air circulation for the build volume of the additive manufacturing system being necessary for this purpose. By forming the polymer layers in vacuum, heat losses due to convection can also be avoided and thus a too rapid cooling of the component can be prevented. As a result, for example, an improved adhesion of layers and a lower surface roughness can be achieved, cf. S. Maidin et al., Int. J. Appl. Eng. Res. 12 (2017), 4877-4881 and S. Maidin et al., J. Fundam. Appl. Sci. 10 (2018) 633-645.

The heat losses due to convection can be controlled by adapting the gas pressure. The gas pressure can be chosen, for example, such that a mean free path λ of the gas particles is similar to or greater than a dimension d of the vacuum chamber in which the method is carried out. In the free molecular flow regime at a Knudsen number of Kn=λ/d>0.5, the gas particles can move almost freely from one wall of the vacuum chamber to another wall of the vacuum chamber and heat transport is independent of pressure. In the viscous flow regime at a Knudsen number of Kn=λ/d<0.01, i.e. a mean free path which is significantly smaller than the dimension of the vacuum chamber, the movement of the gas particles is determined by collisions between the gas particles and heat transport is pressure-dependent. The Knudsen number can be calculated based on the pressure in the vacuum chamber:

$$Kn = \frac{{k}_{B} T}{π {d}_{{N}_{2}}^{2} dp}$$

wherein k_{B} is the Boltzmann constant, T is the temperature and d_{N}_{₂} is the diameter of a nitrogen molecule. The first polymer layer, the first coating layer and the second polymer layer can be formed in the viscous flow regime, in the free molecular flow regime or in a transition regime between the viscous and free molecular flow regimes. The first polymer layer, the first coating layer and the second polymer layer are preferably formed at a Knudsen number of Kn>0.01. In some examples, the corresponding layers can be formed at a Knudsen number of 0.01≤Kn≤1, for example for regulating heat transport. In some examples, the corresponding layers can alternatively also be formed at a Knudsen number of Kn>1 if heat transport by convection is to be prevented.

The invention further provides a device for producing a coated object using the method according to the invention according to any one of the embodiments described herein. The device comprises a vacuum chamber with a receiver configured to receive a workpiece during the production of the coated object. The device further comprises an additive manufacturing unit arranged completely or partially in the vacuum chamber and configured to selectively form a polymer layer on at least a part of the workpiece received by the receiver and/or on at least a part of a printing bed. In addition, the device comprises a coating unit arranged completely or partially in the vacuum chamber and configured to form a coating layer on at least a part of the workpiece and/or of the polymer layer by vacuum-based vapor deposition.

The vacuum chamber can enclose a build volume and comprise a door or a loading lock, e.g., to bring the workpiece into the build volume or to remove the coated object from the build volume. The receiver can be configured to hold the workpiece in a predetermined position during the additive manufacturing process, and can comprise, e.g., a printing bed such as a carrier plate on which the workpiece can be arranged, or a holder in which the workpiece or a printing bed can be mounted, e.g., clamped, by one or more holding means. In particular, the workpiece can have been manufactured in the vacuum chamber by forming a plurality of polymer and/or coating layers in or on the receiver, e.g., on the printing bed, by means of the additive manufacturing unit and/or of the coating unit.

The coating unit is configured to form one or more coating layers on the workpiece by vapor deposition, e.g., on at least a part of the polymer layer formed by the additive manufacturing unit. In particular, the coating unit can be configured to form one or more structured coating layers on the workpiece, i.e., to form the respective layer selectively in one or more regions on the surface of the workpiece. Alternatively or additionally, the coating unit can be configured to form the one or more coating layers homogeneously over the entire workpiece, i.e., over the entire exposed surface of the workpiece. The coating unit can be arranged completely or partially within the vacuum chamber. The coating unit can be arranged, for example, on or in a wall of the vacuum chamber, e.g., on a flange of the vacuum chamber. Preferably, the coating unit is movably supported within the vacuum chamber. The coating unit can be configured to form the one or more coating layers on the workpiece while the workpiece is received by the receiver, e.g., arranged on the printing bed. Alternatively, the coating unit can also be configured to form the one or more coating layers on the workpiece while the workpiece is not received by the receiver, but is located, for example, in another part of the vacuum chamber, e.g., received by another receiving device.

Preferably, the coating unit is configured to form the one or more coating layers by physical vapor deposition, in particular by cathodic arc deposition. The coating unit can comprise, for example, a vaporizer configured to vaporize at least in part a target made of the coating material, e.g., by means of a heating element or a laser source. The coating unit can comprise, in particular, a cathodic arc vaporizer configured to vaporize at least in part a target made of the coating material by means of an electric arc, wherein the coating material is an electrically conductive material or comprises such a material. The cathodic arc vaporizer can be configured to apply an electrical voltage between the target and an electrode in order to form an electric arc between the target and the electrode that locally brings material from the target into the vapor phase.

The coating unit can further be configured to selectively apply the vaporized coating material to the workpiece, e.g., to at least a part of the polymer layer formed by the additive manufacturing unit and/or to at least a part of a part of the workpiece not covered by the polymer layer. For this purpose, the coating unit can be configured to selectively expose the workpiece to the vaporized material. The coating unit can be configured, in particular, to apply the vaporized coating material to a deposition region with a predetermined shape and size on the workpiece. The deposition region can be circular, for example, and can have a diameter between 0.2 mm and 300 mm. The coating unit can be configured, for example, to selectively expose the deposition region on the surface of the workpiece to the vaporized material. The coating unit can comprise, for example, one or more apertures configured to shade one or more regions of the workpiece, e.g., all regions outside the deposition region. The deposition region can have, for example, a diameter between 0.2 mm and 20 mm, in one example a diameter between 0.2 mm and 5 mm. Alternatively or additionally, the coating unit can comprise one or more electrodes and/or one or more magnets configured to shape a beam from the vaporized material and/or direct the beam in a targeted/guided manner onto the workpiece, e.g., onto the deposition region, by means of electromagnetic fields.

The additive manufacturing unit is configured to form one or more structured polymer layers on the workpiece received by the receiver and/or on the printing bed, i.e., to form the respective layer selectively in one or more regions on the surface of the workpiece or of the printing bed, in order to produce a polymer layer with a predetermined shape. In particular, the additive manufacturing unit can be configured to form the one or more polymer layers from a thermoplastic material, e.g., by selective laser sintering (SLS) or selective laser melting (SLM).

In a preferred embodiment, the additive manufacturing unit is configured to form the one or more polymer layers by fused filament fabrication. The additive manufacturing unit can comprise, for example, one or more print heads each arranged in the vacuum chamber and each configured to completely or partially melt a thermoplastic material and to selectively deposit the completely or partially melted thermoplastic material, e.g., at a predetermined position, on the workpiece received by the receiver and/or on the printing bed. Each print head can be configured to receive a filament or a strand of the thermoplastic material and can comprise a heating device to completely or partially melt the filament or strand. Each print head can further comprise a nozzle to deposit the completely or partially melted filament or the completely or partially melted strand in a selective manner at a predetermined position on the workpiece.

The additive manufacturing unit can be arranged completely or partially within the vacuum chamber. In particular, the print head(s) can be arranged within the vacuum chamber. Preferably, each of the print heads is embodied without plastic components, e.g., in order to prevent outgassing in vacuum. The print head(s) can be produced, e.g., completely or partially from metal. In some examples, parts of the additive manufacturing unit can also be arranged outside the vacuum chamber, e.g., a material supply from which the plastic filament is fed to the print head or a laser source for melting the thermoplastic material.

In a preferred embodiment, the receiver, the additive manufacturing unit and/or the coating unit are movably supported within the vacuum chamber, e.g., such that the corresponding element can be moved along at least one direction, preferably along at least two directions. In order to ensure vacuum compatibility, lubricant-free bearings are preferably used in this case. The device can be configured, e.g., to move at least the additive manufacturing unit and the receiver relative to each other, e.g., by moving the additive manufacturing unit and/or the receiver. The device can comprise a drive system configured to move the additive manufacturing unit and/or the coating unit relative to the workpiece received by the receiver and/or to move the workpiece received by the receiver relative to the additive manufacturing unit and the coating unit, preferably along two or three directions. In one example, the device comprises a drive system configured to move the additive manufacturing unit and the coating unit together relative to the workpiece received by the receiver. The additive manufacturing unit and the coating unit can be arranged, e.g., together on or in a carriage and the drive system can be configured to move the carriage, e.g., by means of a motor and a worm gear and/or a belt gear. The drive system can also comprise a guide system with one or more guiding means such as rails and/or movable arms in order to move the carriage along a predefined path. The guiding system can comprise, e.g., Cartesian kinematics or delta kinematics.

In some examples, the device further comprises a control unit configured to control a thickness of the coating layer, preferably by adjusting a duration of the vapor deposition. The control unit can be implemented as hardware and/or software and can comprise, e.g., a processor and a storage medium, wherein the storage medium stores instructions that can be executed by the processor to provide the functionality described herein. The control unit can be configured, e.g., to control the coating unit and/or a drive system for the coating unit, for example to adjust a vaporization time during which the coating unit vaporizes coating material and/or a dwell time and/or speed of the coating unit over and relative to, respectively, a region of the workpiece.

The device can also comprise a cooling system configured to cool the receiver, the additive manufacturing unit, the coating unit and/or a drive system of the device, e.g., by circulating a heat exchange medium through and/or around the corresponding components, e.g., water. The cooling system can be connected to the corresponding components by means of vacuum-compatible tubes and can comprise a reservoir for the heat exchange medium and a pump configured to circulate the heat exchange medium through and/or around the corresponding components via the tubes. Thereby, parts within the vacuum chamber that run hot can be cooled actively, e.g., a print head of the additive manufacturing unit, a vaporizer of the coating unit, one or more motors of the drive system and/or components adjacent to these parts. This can be advantageous for operating the corresponding components in vacuum, since no or hardly any heat transport by convection takes place in vacuum. In addition, the workpiece received in the receiver can also be cooled directly or indirectly. The cooling system can be controlled by the control unit, for example.

The device can further comprise a pressure control system configured to set a predetermined gas pressure within the vacuum chamber. The predetermined gas pressure can be less than 1 mbar, in some examples less than 10⁻¹ mbar, preferably less than 10⁻² mbar, in one example less than 10⁻³ mbar and in one example less than 10⁻⁴ mbar. The pressure control system can be configured, for example, to set the gas pressure within the vacuum chamber to a value between 10⁻¹ mbar and 1 bar, preferably between 10⁻⁴ mbar and 1 bar. For this purpose, the pressure control system can comprise, for example, a vacuum pump and a pressure sensor, wherein the control unit can be configured to control the vacuum pump depending on the pressure measured by the pressure sensor.

### BRIEF DESCRIPTION OF THE DRAWINGS

In the following, the invention is explained in more detail on the basis of exemplary embodiments with reference to the accompanying drawings. The figures show schematic illustrations of:

FIG. 1: a device for producing a coated object according to an exemplary embodiment of the invention;

FIG. 2: a flowchart of a method for producing a coated object according to an exemplary embodiment of the invention;

FIG. 3a: the formation of a first polymer layer on a workpiece according to an exemplary embodiment of the invention;

FIG. 3b: the formation of a first coating layer on the workpiece from FIG. 3a according to an exemplary embodiment of the invention;

FIG. 3c: the formation of a second polymer layer on the workpiece from FIG. 3b according to an exemplary embodiment of the invention;

FIG. 4a: the formation of a second polymer layer on a workpiece according to a further embodiment of the invention; and

FIG. 4b: the formation of a first coating layer on a workpiece according to a further embodiment of the invention.

### DESCRIPTION OF THE DRAWINGS

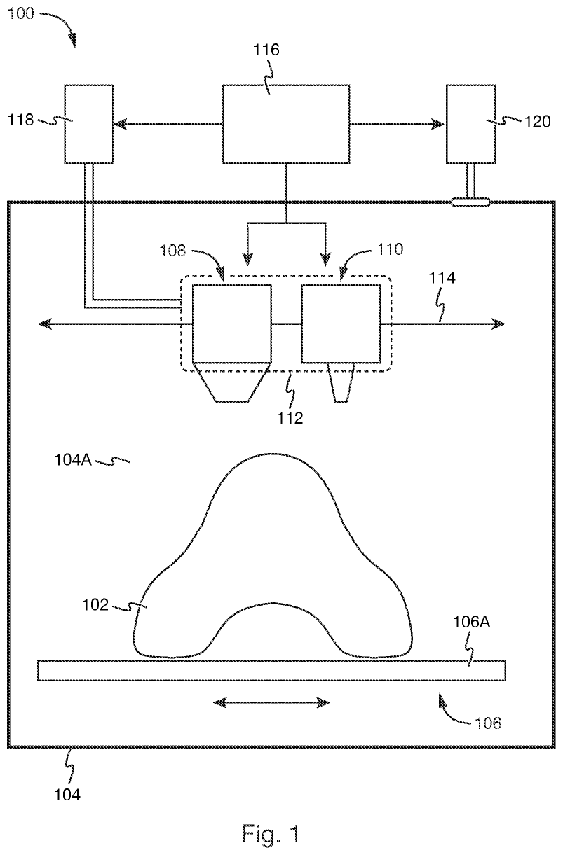

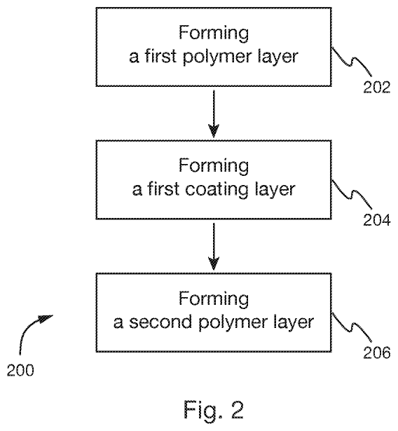

FIG. 1 shows a schematic illustration (not to scale) of a device 100 for producing a coated object 102 according to an exemplary embodiment of the invention. The device 100 can in particular be used for producing a coated object using a method for producing a coated object according to any one of the embodiments described herein, for example to carry out the method 200 described below with reference to FIG. 2.

The coated object 102 can be a medical implant such as a bone replacement implant, which is formed with an osseointegrative and/or antibacterial coating. However, both the device 100 and the method 200 are not limited to this example and can for example alternatively or additionally also be used to produce a component, e.g., for use in aerospace, wherein the component is formed with a functionalized coating, e.g., with a strengthening reinforcement, an electrically conductive coating or a thermally conductive coating. In other examples, the device 100 and/or the method 200 can also be used to form a component with an optical coating, e.g., a reflective coating, an antireflection coating or a coating with a particular color.

The device 100 comprises a vacuum chamber 104 which encloses a build volume 104A and is configured to maintain a vacuum therein. The vacuum chamber 104 can be made, e.g., from metal, for example stainless steel, and can comprise a door or a loading lock (not shown) in order to bring a workpiece into the build volume 104A for producing the coated object 102 or to remove the finished coated object 102 from the build volume 104A.

A receiver 106 is arranged in the vacuum chamber 104 and is configured to receive the workpiece during the production of the coated object 102. In the example of FIG. 1, the receiver 106 comprises a printing bed 106A, e.g., a carrier plate, on which the workpiece or the coated object 102 can be arranged and held. The receiver 106 can further comprise one or more holding means (not shown) configured to mount, e.g., clamp, the workpiece or the coated object 102 on the carrier plate. The receiver 106 can be movably supported within the vacuum chamber 104, e.g., such that the receiver 106 can be moved along two or three spatial directions. In other embodiments, the receiver 106 can be fixedly installed in the vacuum chamber 104 and can be mounted, e.g., to a wall of the vacuum chamber 104.

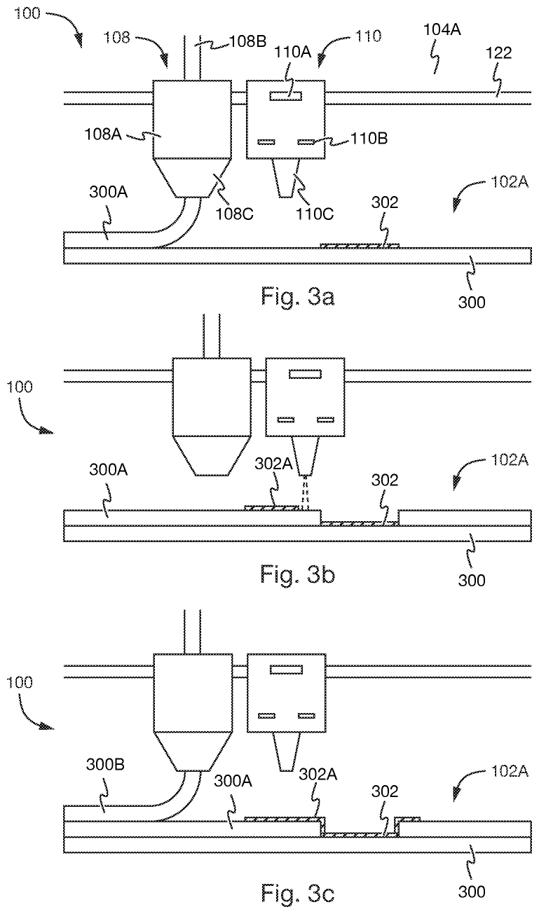

The device 100 further comprises an additive manufacturing unit 108 configured to selectively form a polymer layer (not shown) on at least a part of a workpiece arranged on or in the receiver 106 and/or on at least a part of the printing bed 106A. The additive manufacturing unit 108 can be configured, e.g., to deposit the polymer layer of a thermoplastic material such as polyetheretherketone (PEEK) on the workpiece by fused filament fabrication (fused deposition modeling), as explained below with reference to FIG. 3a.

The device 100 further comprises a coating unit 110 configured to form a coating layer (not shown) of a coating material such as an osseointegrative material, e.g., titanium, on the workpiece by vacuum-based vapor deposition. The coating unit 110 can be configured, e.g., to deposit the coating layer on the workpiece by cathodic arc deposition, as explained below with reference to FIG. 3b.

In the example of FIG. 1, the additive manufacturing unit 108 and the coating unit 110 are arranged on a movably supported carriage 112 which can be moved along a predefined path 114 in at least two spatial directions, preferably in all three spatial directions. The device 100 can comprise, e.g., a drive system (not shown) configured to move the carriage 112 along the path 114. The drive system can comprise, e.g., one or more motors configured to drive the carriage 112 and a guiding system configured to guide the carriage 112 along the path 114, e.g., as explained below with reference to FIG. 3a.

The device 100 further comprises a control unit 116 configured to control the additive manufacturing unit 108, the coating unit 110 and the carriage 112. The control unit 116 can comprise, e.g., a microcontroller (not shown) having a processor and a storage medium, wherein the storage medium contains instructions for execution by the processor to provide the functionality described herein. The control unit 116 is configured to control the deposition of polymer material by the additive manufacturing unit 108, the deposition of the coating material by the coating unit 110 and the movement of the carriage 112. In particular, the control unit 116 is configured to control a thickness of the coating layer formed by the coating unit 110 by adjusting a duration of the vapor deposition. For this purpose, the control unit 116 can selectively switch the coating unit 110 on and off and/or adjust a speed or dwell time of the carriage 112 during the formation of the coating layer. Alternatively or additionally, the control unit 116 can also be configured to adjust a coating rate of the coating unit 110, e.g., by adjusting a vaporization rate of the coating material. In some examples, the control unit 116 can be configured to carry out the method 200 in its entirety or in part.

The device 100 further comprises a cooling system 118 configured to cool the drive system of the device 100, in particular the motors of the drive system, e.g., by providing a circulating coolant. Thereby, operation of the drive system in vacuum, in which no or hardly any heat can be released by convection, can be made possible. The cooling system 118 is further configured to cool the additive manufacturing unit 108, the coating unit 110, the carriage 112 or parts thereof. In some embodiments, the cooling system 118 can alternatively or additionally also be configured to cool the receiver 106, the workpiece, the coated object 102 and/or the build volume 104A. The control unit 116 is configured to control the cooling system 118 and to for example adjust a flow rate and/or a temperature of the coolant.

The device 100 further comprises a pressure control system 120 configured to set a predetermined gas pressure in the build volume 104A. For this purpose, the pressure control system 120 can comprise, for example, a vacuum pump (not shown) connected to a flange or a valve of the vacuum chamber 104. The pressure control system 102 can also comprise a gas control valve (not shown) configured to allow gas from the environment of the vacuum chamber 104 to flow into the build volume 104A. The pressure control system 120 can further comprise a pressure sensor (not shown) configured to determine the gas pressure in the build volume 104A. The control unit 116 can be configured to control the vacuum pump and/or the gas control valve of the pressure control system 120 depending on the gas pressure measured by the pressure sensor to regulate the gas pressure to a predetermined target value. Preferably, the pressure control system 120 is configured to set the gas pressure in the build volume 104A within a range between 10⁻³ mbar and 1 bar, in one example between 10⁻⁴ mbar and 1 bar and in one example between 10⁻⁵ mbar and 1 bar.

FIG. 2 shows a flowchart of a method 200 for producing a coated object such as the coated object 102, for example a medical implant with an osseointegrative and/or antibacterial coating, by additive manufacturing according to an exemplary embodiment of the invention. The method 200 can be carried out, for example, with a device according to the invention in accordance with any one of the embodiments described herein. In the following, the method 200 is described by way of example on the basis of the device 100 from FIG. 1. The method 200 can be carried out in its entirety or in part by a control unit of the corresponding device, for example the control unit 116 of the device 100. The execution of the method 200 is not limited to the sequential order indicated by the flowchart in FIG. 2. As far as technically feasible, the steps of the method 200 can also be carried out simultaneously at least in part. For example, the formation of the first coating layer in step 204 can be started before the formation of the first polymer layer in step 202 has been completed. In particular, the second polymer layer can be formed before, simultaneously with or after the first coating layer. For example, the formation of the second polymer layer in step 206 can be started before the formation of the first coating layer in step 204 has been completed. For this purpose, the additive manufacturing unit 108 and the coating unit 110 of the device 100 can be activated simultaneously, for example.

In FIGS. 3a, 3b and 3c, individual steps of the method 200 are illustrated schematically. In these examples, the additive manufacturing unit 108 is configured to form polymer layers 300, 300A of a thermoplastic material, e.g., polyetheretherketone (PEEK), on a workpiece 102A arranged in the receiver 106 by fused filament fabrication. For this purpose, the additive manufacturing unit 108 comprises a print head 108A to which a filament 108B of the thermoplastic material is fed. The print head 108A is configured to at least partially melt the filament 108B and to deposit the melted plastic through a nozzle 108C in a selective manner at a predetermined position on the workpiece 102A. Here, a workpiece 102A refers to the coated object 102 in a precursor state at the beginning and during the manufacturing of the coated object 102. In particular, before the start of the method 200 or as part of the method 200 before forming the first polymer layer 300A in step 202, the workpiece 102A can be additively manufactured on the printing bed 106A using the device 100, e.g., by forming a plurality of polymer and/or coating layers. In other embodiments, the first polymer layer 300A in step 202 can also be formed directly on the printing bed 106A instead of on the workpiece 102A and, for example, can be the very first layer of the coated object 102, i.e., before forming the first polymer layer 300A, no workpiece 102A can have been provided or manufactured on the printing bed 106A.

Furthermore, in these examples, the coating unit 110 is configured to form coating layers 302, 302A of a coating material such as an osseointegrative material, e.g., titanium, on the workpiece 102A by cathodic arc deposition. For this purpose, the coating unit 110 comprises a cathodic arc vaporizer configured to receive a target 110A made of the coating material and to apply a voltage between the target 110A and one (or more) electrodes 110B to generate an electric arc (not shown) from the electrode 110B to the target 110A by means of a vacuum arc discharge process. The electric arc can remove coating material at a surface of the target 110A and bring it into an almost completely ionized state. The removed material is almost completely ionized and can be accelerated by an additional voltage applied between the electrode 110B and the workpiece 102A and/or the printing bed 106A. By means of an aperture or nozzle 110C, the workpiece 102A can be partially shaded and exposed to the vaporized coating material in a selective manner at a predetermined position only, e.g., by generating a conically expanding beam of the coating material as shown schematically in FIG. 3b. In some examples, the beam of coating material can also be substantially collimated.

The additive manufacturing unit 108 and the coating unit 110 can be arranged on a carriage (not shown in FIGS. 3a-3c) as shown in FIG. 1 and can be moved along one or more guide rails 122 of a guiding system in the build volume 104A using a drive system. The guiding system can comprise, e.g., Cartesian kinematics in which the guide rails 122 extend along a plurality of orthogonal directions. Alternatively or additionally, the guiding system can also comprise delta kinematics with one or more rotatably supported and/or retractable/extendable arms. In other embodiments, the device 100 can also be configured to move the additive manufacturing unit 108 and the coating unit 110 independently of one another and/or to move the receiver (not shown in FIGS. 3a-3c) for the workpiece 102A relative to the additive manufacturing unit 108 and the coating unit 110.

At the beginning of the method 200, a workpiece 102A is provided in the receiver 106. The workpiece 102A can be made, e.g., from plastic, ceramic and/or metal and can form, e.g., a base frame for the component 102. In some embodiments, the workpiece 102A can be produced completely or partially by additive manufacturing before the start of the method 200 or as part of the method 200. For this purpose, a plurality of polymer layers 300 and/or a plurality of coating layers 302 can be iteratively formed, e.g., with the additive manufacturing unit 108 and/or the coating unit 110, wherein only one polymer layer 300 and one coating layer 302 are shown in FIGS. 3a-3c for the sake of simplicity. The polymer layers 300 and the coating layers 302 can be formed, e.g., directly on the printing bed 106A of the receiver 106 or on a substrate arranged in the receiver 106 or on the printing bed 106A. In some examples, a base frame (not shown) for the workpiece 102A produced by other means can be provided in the receiver 106 and the polymer layers 300 and the coating layers 302 can be formed on the base frame. The base frame can comprise, e.g., plastic, ceramic and/or metal and can have been produced, e.g., by milling and/or injection molding.

The method 200 can further comprise evacuating the build volume 104A before forming the first polymer layer 300A in step 202, e.g., after providing the workpiece 102A or before additively manufacturing the workpiece 102A. For this purpose, the vacuum chamber 104 can be pumped down, e.g., by means of the pressure control system 120, until a predetermined gas pressure is reached. The predetermined gas pressure can be, e.g., between 10⁻⁵ mbar and 1 mbar, preferably between 10⁻⁴ mbar and 0.1 mbar. In one example, the predetermined gas pressure is between 0.5·10⁻⁴ mbar and 2.0·10⁻⁴ mbar, e.g., 1.0·10⁻⁴ mbar. The gas pressure in the vacuum chamber 104 can be kept constant during the entire method 200, in particular during the execution of steps 202, 204 and 206. In some embodiments, the build volume 104A can already have been evacuated before the start of the method 200 and the workpiece 102A or a base frame therefor can be arranged in the receiver 106, where appropriate, via a loading lock (not shown).

The method 200 first comprises forming a first polymer layer 300A on the workpiece 102A in step 202, e.g., as shown in FIG. 3a. For this purpose, the print head 108A is moved along a predefined path by means of the guide rails 122 and molten plastic is selectively deposited on the workpiece 102A in the meantime to form the first polymer layer 300A. The shape of the first polymer layer 300A can be determined, e.g., on the basis of a construction model for the coated object 102, e.g., a CAD model, and the melted plastic can be deposited accordingly on the workpiece 102A by suitably selecting the path and/or activating the print head 108A, e.g., to form a structured first polymer layer 300A as shown in FIG. 3b. A thickness of the first polymer layer 300A can be, e.g., between 0.05 mm and 2 mm, preferably between 0.1 mm and 1 mm. In some embodiments, forming the first polymer layer 300A in step 202 can also comprise a cooling phase in which the deposited plastic can solidify.

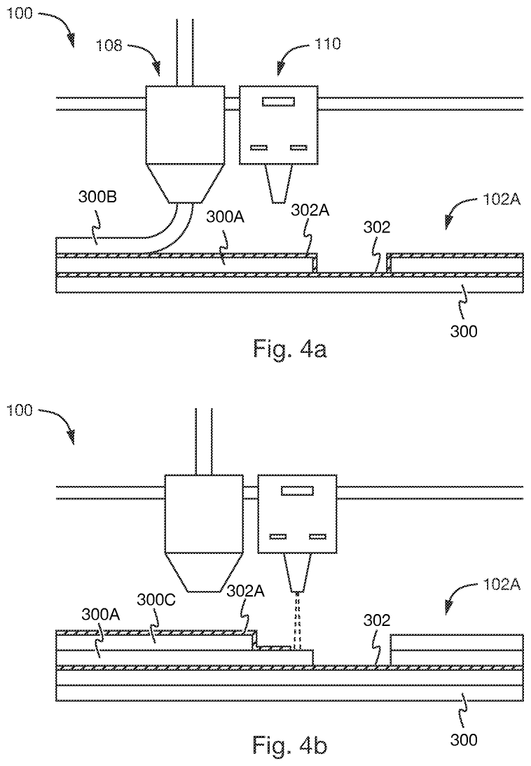

Subsequently, in step 204, a first coating layer 302A is formed from the coating material on the first polymer layer 300A, e.g., as shown in FIG. 3b. For this purpose, the coating unit 110 is moved along a predefined path by means of the guide rails 122 and the coating material is selectively deposited on the workpiece 102A by vacuum-based vapor deposition in the meantime, e.g., by cathodic arc deposition. In a preferred embodiment, the first coating layer 302A is formed only on those regions of the first polymer layer 300A which form an exposed surface of the coated object 102 after completion of the production of the coated object 102, while no coating material is deposited on regions of the first polymer layer 300A which are later covered by one or more further polymer layers, e.g., the second polymer layer 300B. In other embodiments, the first coating layer 302A can also be formed on at least a part of the regions of the first polymer layer 300A which are later covered by one or more further polymer layers, e.g., on edge regions abutting the later exposed regions or on the entire first polymer layer, e.g., as shown in FIG. 4a. The first coating layer 302A can be formed, e.g., with a thickness between 1 nm and 10 μm, preferably between 50 nm and 500 nm, in one example with a thickness of 100 nm.

After forming the first coating layer 302A, a second polymer layer 300B is formed on the workpiece 102A in step 206, e.g., as shown in FIG. 3c. The second polymer layer 300B is formed similarly to the first polymer layer 300A in step 202, e.g., by selectively depositing molten plastic based on a CAD model of the coated object 102A. In a preferred embodiment, the second polymer layer 300B is formed only on exposed regions of the first polymer layer 300A which are not covered by the first coating layer 302A. In other embodiments, the second polymer layer 300B can also be formed already before forming the first coating layer 302A or during the formation of the first coating layer 302A.

The method 200 can also comprise iteratively forming further polymer layers and/or further coating layers after forming the second polymer layer 300B in step 206, e.g., until the desired shape of the coated object 102 is obtained. In particular, the method 200 can comprise forming a second coating layer (not shown) of the coating material on at least a part of the second polymer layer 300B by vacuum-based vapor deposition.

In some embodiments, the first coating layer 302A can be formed uniformly on the entire first polymer layer 300A, as shown in FIG. 4a, e.g., by coating the entire exposed surface of the workpiece 102A with the coating material. In some examples, the coating unit 110 can, e.g., be fixedly installed in the vacuum chamber 104 instead of on the carriage 112 and can be configured to apply the vaporized coating material to the workpiece 102A over a large area instead of a conically expanding or substantially collimated beam of the coating material as shown in FIG. 3b. Alternatively, the first coating layer 302A can also be applied homogeneously onto the entire first polymer layer 300A by suitably moving the coating unit 110.

The polymer layers 300, 300A, 300B and the coating layers 302, 302A can be formed in an alternating order, as shown in FIGS. 3a-3c and 4a, i.e., a respective coating layer can be formed between two successive polymer layers each. In some embodiments, the coated object 102 can also be produced completely or partially using one or more other sequences of layers. For example, a base frame for the coated object 102 can first be produced which does not comprise any coating layers, but rather consists, e.g., exclusively of polymer layers and/or other materials, for example because none of the respective parts of the coated object 102 are exposed after completion of the production of the coated object 102. Subsequently, parts of the coated object 102 that are close to the surface can be formed, e.g., in an alternating layer sequence of polymer layers and coating layers. In some embodiments, only a single coating layer can be formed on the coated object 102, e.g., at the end of the production after all polymer layers for the coated object 102 have been formed.

Alternatively or additionally, at least parts of the coated object 102 can be formed in a sequence of layers comprising fewer coating layers than polymer layers. For example, a predetermined number of polymer layers, e.g., between 2 and 10 polymer layers, can first be formed before a coating layer is formed on these polymer layers, e.g., on the regions of these polymer layers that are exposed after completion of the production of the coated object. Subsequently, the predetermined number of polymer layers and then a further coating layer can again be formed. In FIG. 4b, this is shown by way of example for a sequence of layers in which two polymer layers are formed per coating layer. For this purpose, after or simultaneously with the formation of the first polymer layer 300A, a further polymer layer 300C is first formed before the first coating layer 302A is formed on at least a part of the first polymer layer 300A and/or on at least a part of the further polymer layer 300C.

Due to the additive manufacturing unit 108 and coating unit 110 arranged in the vacuum chamber 104, the device 100 enables the formation of any layer sequences of polymer and/or coating layers in a common manufacturing process without the workpiece 102A having to be removed from the vacuum chamber 104. Thus, objects with complex structures and one or more coatings can be formed, wherein the coatings can be formed on a surface of the object as well as in the interior of the object. In some examples, the device can comprise, in addition to the additive manufacturing unit 108 and the coating unit 110, one or more further additive manufacturing units and/or one or more further coating units, e.g., to form polymer layers of different materials and/or coating layers of different materials.

The embodiments according to the invention described here and the figures serve for purely exemplary illustration only. The invention can vary in its form without changing the underlying functional principle. The scope of protection of the device according to the invention and the method according to the invention is solely defined by the appended claims.

### LIST OF REFERENCE SIGNS

100—Device for producing a coated object
102—Coated object
104—Vacuum chamber
104A—Build volume
106—Receiver
106A—Printing bed
108—Additive manufacturing unit
108A—Print head
108B—Plastic filament
108C—Nozzle
110—Coating unit
110A—Target
110B—Electrode
110C—Aperture/nozzle
112—Carriage
114—Printing path
116—Control unit
118—Cooling system
120—Pressure control system
122—Guide rail
200—Method for producing a coated object
202—Step of forming a first polymer layer
204—Step of forming a first coating layer
206—Step of forming a second polymer layer
300—Polymer layer
300A—First polymer layer
300B—Second polymer layer
300C—Further polymer layer
302—Coating layer
302A—First coating layer

What is claimed is:

1.-20. (canceled)

21. A method for producing a coated object by additive manufacturing in vacuum, the method comprising:
forming a first polymer layer on one or both of a workpiece and a printing bed;
forming a first coating layer on at least a part of the first polymer layer by vacuum-based vapor deposition; and
forming a second polymer layer on at least a part of one or both of the first polymer layer and the first coating layer, wherein the second polymer layer is formed before, during or after forming the first coating layer,
wherein the first polymer layer, the first coating layer and the second polymer layer are formed in vacuum at a gas pressure of less than 1 mbar.

22. The method according to claim 21, wherein the first coating layer comprises a material that is one or more of inorganic, thermally conductive and electrically conductive.

23. The method according to claim 21, wherein the coated object is a medical implant and the first coating layer comprises a material that is one or both of osseointegrative and antibacterial.

24. The method according to claim 21, wherein the first coating layer is formed by physical vapor deposition.

25. The method according to claim 21, wherein the gas pressure in the vacuum when forming the first polymer layer, the first coating layer and the second polymer layer is less than 10⁻² mbar.

26. The method according to claim 21, wherein the first coating layer is formed only on regions of the first polymer layer which form an exposed surface of the coated object after completion of the production of the coated object.

27. The method according to claim 21, wherein forming the first coating layer comprises adding a process gas for forming a reactive coating.

28. The method according to claim 21, wherein the first polymer layer and the second polymer layer each comprise one or both of an engineering polymer and a high-performance polymer.

29. The method according to claim 21, wherein the first polymer layer and the second polymer layer are each formed by fused filament fabrication.

30. The method according to claim 21, further comprising additively manufacturing the workpiece by one or both of:
iteratively forming a plurality of polymer layers; and
iteratively forming a plurality of coating layers by vacuum-based vapor deposition.

31. A device for producing a coated object using a method for producing the coated object by additive manufacturing in vacuum, the method comprising:
forming a first polymer layer on one or both of a workpiece and a printing bed;
forming a first coating layer on at least a part of the first polymer layer by vacuum-based vapor deposition; and
forming a second polymer layer on at least a part of one or both of the first polymer layer and the first coating layer, wherein the second polymer layer is formed before, during or after forming the first coating layer and wherein the first polymer layer, the first coating layer and the second polymer layer are formed in vacuum at a gas pressure of less than 1 mbar,
the device comprising:
a vacuum chamber with a receiver configured to receive the workpiece during the production of the coated object;
an additive manufacturing unit arranged completely or partially in the vacuum chamber and configured to selectively form a polymer layer on at least a part of one or both of the workpiece received by the receiver and the printing bed; and
a coating unit arranged completely or partially in the vacuum chamber and configured to form a coating layer on at least a part of one or both of the workpiece and the polymer layer by vacuum-based vapor deposition.

32. The device according to claim 31, wherein the coating unit comprises a cathodic arc vaporizer configured to vaporize at least in part a target made of a conductive material by means of an electric arc.

33. The device according to claim 32, wherein the coating unit is configured to selectively apply the vaporized conductive material to one or both of the workpiece and the polymer layer.

34. The device according to claim 31, wherein the additive manufacturing unit comprises a print head arranged in the vacuum chamber and configured to completely or partially melt a thermoplastic material and to selectively deposit the completely or partially melted thermoplastic material on one or both of the workpiece received by the receiver and the printing bed.

35. The device according to claim 31, wherein one or more of the receiver, the additive manufacturing unit and the coating unit are movably supported within the vacuum chamber.

36. The device according to claim 35, further comprising a drive system that is one or both of:
configured to move one or both of the additive manufacturing unit and the coating unit relative to the workpiece received by the receiver; and
configured to move the workpiece received by the receiver relative to the additive manufacturing unit and the coating unit.

37. The device according to claim 36, wherein the drive system is configured to move the additive manufacturing unit and the coating unit together relative to the workpiece received by the receiver.

38. The device according to claim 31, further comprising a control unit configured to control a thickness of the coating layer.

39. The device according to claim 31, further comprising a cooling system configured to cool one or more of the receiver, the additive manufacturing unit, the coating unit and a drive system of the device.

40. The device according to claim 31, further comprising a pressure control system configured to set a predetermined gas pressure within the vacuum chamber, wherein the predetermined gas pressure is less than 1 mbar.
